(19) United States
(12) Patent Application Publication
(10) Pub. No.: US 2009/0043397 A1
(43) Pub. Date: Feb. 12, 2009

(54) Modular Femoral Head Surface Replacement, Modular Femoral Neck Stem, and Related Sleeve, Adapter, and Osteoconducting Rod

(76) Inventor: Hyung-bae Park, Jeonju (KR)

Correspondence Address: PARK LAW FIRM, 3255 WILSHIRE BLVD, SUITE 1110, LOS ANGELES, CA, 90010, US

(21) Appl. No.: 11/837,346

(22) Filed: Aug. 10, 2007

(51) Int. Cl. A61F 2/36

(52) U.S. Cl. 623/23.11; 623/23.12; 623/23.15

(57) ABSTRACT

Disclosed therein is a big femoral head and femoral head surface replacement, which are used for hip osteoarthritis and vascular necrosis of femoral head. As a person get older and aged, the weight bearing hip joint is indispensably changed to osteoarthritis and sometimes showed avascular necrosis of the femoral head with unknown etiology. The deformed femoral head and hip joint should be replaced with the THA. Till now, the total hip replacement is performed in such a way that the necrosed femoral head and a healthy femoral neck are all removed and a femoral stem is inserted into the marrow cavity, and in this case, a small femoral head causes a reduction of a range of motion and dislocation of the hip joint occasionally, and osteolysis due to abrasion of plastic acetabular liner. In case of a conventional femoral head surface replacement (hereinafter, called “conventional FHSR”), a complication of femoral neck fracture and could not combined use with conventional THA. Recently, hard bearing system such as metal on metal THA or ceramic on ceramic THA without using plastic has been introduced to solve the problem of osteolysis due to abraded plastic particles generated when the THA is worn out as time goes. But there also have many problems as a limited range of motion, resected normal femoral neck and difficulties of rereplacement of the femoral stem. Because of the big femoral head or the FHSR can increase the range of motion and lower dislocation rate these devices are gradually widespread in young active person and Asian peoples. This invented design of the modularity gives the convenience to the surgeon and economically lower burden to patients to use of the FHSR and big femoral head system. The related accessory showed initial stability of the FHSR during operation and prevent from femoral neck fracture in follow-up periods.

### BACKGROUND OF THE INVENTION

1. Field of the Invention

The present invention relates to a modular femoral head surface replacement, a modular femoral neck stem, and related sleeve, adapter, and osteoconducting rod, which are used to perform total hip replacement.

2. Background Art

In general, as a person grows older, a cartilage of the femoral head is gradually worn out or disappeared, and so, it may result in osteoarthritis of the hip joint or avascular necrosis of the femoral head (hereinafter, referred to as “AVN”) due to an unknown cause. In the above case, at the early stage, various surgical operations may be performed to preserve the femoral head, but finally, total hip arthroplasty (hereinafter, referred to as “THA”) is performed. Recently, the femoral head surface replacement (hereinafter, called “FHSR”), which had failed in the past, has obtained good results through an innovative development of materials of metal. and so, many companies are producing approvals. In the conventional THA, the size of the femoral head, which was 22 mm previously, has been increased gradually to 28 mm and 32 mm, and recently, the femoral head of more than 36 mm has been experimentally developed and used. Such a big femoral head has several merits in that a wide range of motion, low dislocation rate and it is little wearing since it is a metal on metal FHSR than previous polyethylene on metal THA. However, the big femoral head has several disadvantages as cost of manufacturing is very expensive since several kinds of the femoral head must be manufactured and prepared for each patients. However, a modular femoral head system(a surface replacement or a big femoral head) according to the present invention is economical since it is using a modular locking methods without needing to independently manufacture all kinds of the femoral head, and maximizes convenience in use since the big femoral head can be inserted using an invented adapter for connecting a conventional THA or osteoconducting metal rod

Since a conventional THA are composed of a plastic liner in a metal acetabular cup, and a small metal or ceramic femoral head (diameter of head is commonly 28 mm) in the femoral stem. The conventional THA has several problems, such as frequent dislocation because of small femoral head wearing of plastic liner and resulted osteolysis due to abraded plastic microparticles. To treat the AVN, multiple drillings, vascularized bone graft, or rotational osteotomy can be performed in the initial stage of AVN, but finally, the best surgical treatment is to perform total hip replacement after cutting the necrotic femoral head and femoral neck.

Recently, there has been reported that a successful results from femoral head surface replacement by USA and UK. However, the femoral head surface replacement have some complications such a femoral neck fracture and suspicious metal ions accumulation in the organ. For prevention of the femoral neck fracture is not to make a notching of the femoral neck during operation or a valgus fixation of a femoral neck stem is recommended and but there were no proven harmful reports for metal ions accumulation to this time. The conventional THA, which has been performed to patients in Asian people who have the seating life style, namely, mainly seat on the floor, feel inconvenience in performing house affairs and working at work places, and in that a young sportsman, who underwent the surgery of the conventional THA, cannot live as the sportsman again because of narrow range of motion and earlier wearing of polyethylene liner. However, recently, metal on metal FHSR or a big femoral head using THA comes into the spotlight since ballet dancer, baseball player or golfers can keep their positions as sportsmen after undergoing the surgery with a FHSR or total hip replacement.

### SUMMARY OF THE INVENTION

The total hip replacement, which is the final treatment method of osteoarthritis and AVN of the femoral head, is the most successful treatment to relieve the pain and recover the stability and increase the range of motion. However, as time goes, because of progressive worn-out of a plastic liner, resulted osteolysis in the pelvis and femur by abraded particles, a reduction in a range of motion (96 degree in case of the femoral head of 22 mm, and 103 degree in case of the femoral head of 28 mm) of the hip joint and increased dislocation rate of the hip joint due to a small femoral head and instability. Anyone who managed with conventional THA, showed difficulties in sports activities. A big and hard-bearing femoral head is preferred gradually, and so, a CoCr-alloy or a ceramic femoral head comes into the spotlight. As commonly, simple and fixed-type big femoral heads or femoral head surface replacements are used in the world medical market, but a modular femoral head surface replacement according to the present invention give a convenience and supported to a surgeon to choose a straight or tapered, fluted type stem according to the patients' bone state.

Now is the time to challenge to the market of medical appliances owing to development of metal industry and ceramic technology. The most important point of the surgeon who chooses the implant is the design. The modularity, adapter and sleeve systems are to give a chance to surgeon to correct misdirected located femora stem by using an eccentric femoral neck stem or adapter. The modularity give a forgiveness to surgeon to change the operation methods, the sleeve system gives an initial stability of the femoral stem and the adapter can be used with conventional THA alternatively

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1(A) is a view of a conventional femoral head surface replacement (hereinafter, called “conventional FHSR”) having a femoral head (A1) and a fixe straight type femoral stem (A2).

FIG. 1(B) is a block diagram of a modular femoral head surface replacement (hereinafter, called “modular FHSR”) or a modular big femoral head according to a preferred embodiment of the present invention, wherein a female-type connection portion 1 having a fixing force is formed at the tip of the modular FHSR or the modular big femoral head, to which weight is loaded. The female-type connection portion 1 is maybe manufactured according to surgeon's choice whether a cone-tapered type 1-C or a rectangular-tapered type 1-R.

FIG. 1(C) is a cross sectional view of a conventional big femoral head, in which a connection portion 11 is fixed with the femoral head.

FIG. 2 is a view of a modular femoral neck stem (2) according to a preferred embodiment of the present invention, which has a male-type connection portion formed at the proximal portion thereof, the male-type connection portion adopting one of a conically-tapered type 11-C and a rectangularly-tapered type 11-R and being connected with the female-type connection portion of the modular big femoral head. A hole 12 for allowing an easy insertion and removal is formed on the distal portion of the modular femoral neck stem (2). A shaft of the modular femoral neck stem 2 may adopt one of a straight type 21, a tapered type 22, a fluted type 23 and an eccentric type 24, which has an angle of more than 10 degree, according to differences in size among a person, who has small bones, like an Oriental, and a person, who has big bones, like a Westerner, or according to distinction of sex. The reference numeral 23-c indicates a cross section of the fluted femoral neck stem 23.

FIG. 3 is a view of a sleeve (3) fit on the modular femoral neck stem 2, which may adopt one of a straight type 31, a tapered type 32, a straight fluted type 33 and a tapered fluted type 34.

FIG. 4 is a view of an adapter (4) connected with the femoral neck of the big femoral head and a conventional THA which may adopt one of a straight type 41 and an eccentric type 42, and an adapter 4 connected with an osteoconducting rod (5) used to treat avascular necrosis (hereinafter called, “AVN”) of the femoral head, which may adopt one of a straight type 43 and an eccentric type 44.

FIG. 5 is a sectional view of the osteoconducting rod (5) used to treat AVN, which composed with a container 51 mounted on the tip portion thereof for storing BMP family or hydroxyapatite granules or stem cells, a shaft 52 and screw portion 53. The surface of the shaft 52 which treated with a rough blasted techniques for bone ingrowth. The osteoconducting rod 5 is inserted in the beginning stage 61 of AVN of a femur 6. In FIG. 5, a capsule 54 which can be made of gelatin, polylactic acid or polyglycolic acid, which is naturally dissolved as a long time goes, is illustrated. We have a plan to insert a stem cell into the capsule 54 in the future to stop the progression of the necrosis and revasulize in the collapsed area. In FIG. 5, the right side shows a state where the osteoconducting rod 5 is inserted into the hip joint where necrosis starts. FIG. 5 shows the order to insert an artificial femoral head when necrosis makes progress.

### DETAILED DESCRIPTION OF THE PREFERRED EMBODIMENT

Reference will be now made in detail to the preferred embodiment of the present invention with reference to the attached drawings so that those skilled in the art can easily understand and reproduce.

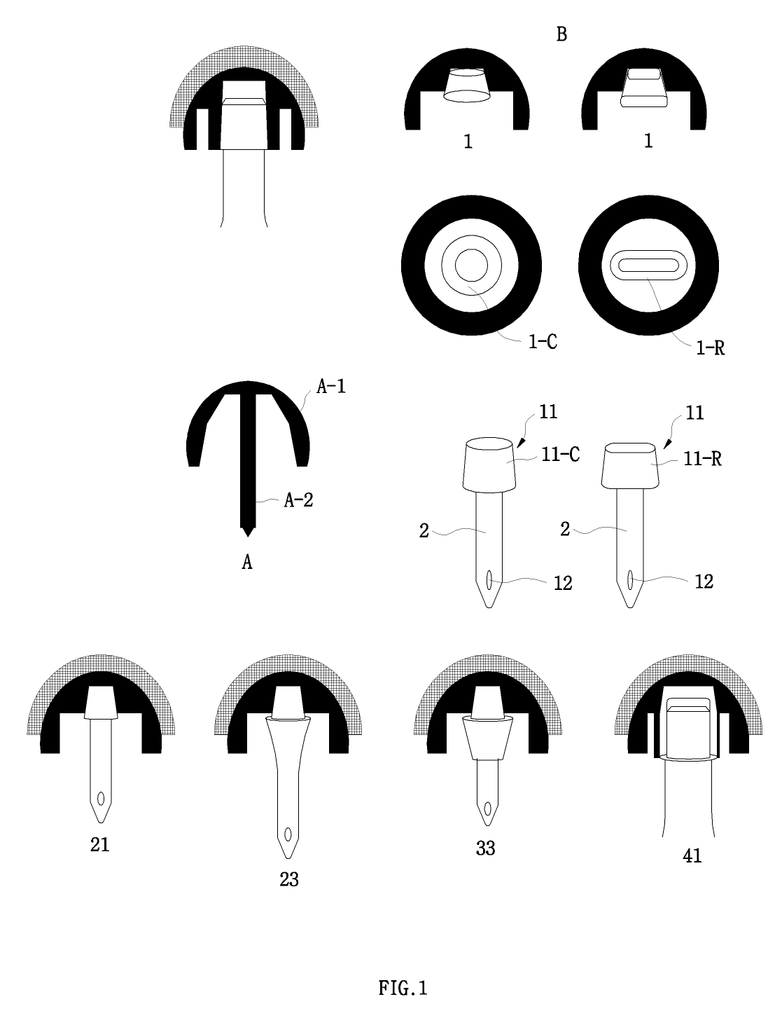

FIG. 1 illustrates a conventional femoral head surface replacement (A) (hereinafter, called “FHSR”) of a fixed one-body type, in which a femoral head (A1) and a neck stem (A2) are fixed thereto. In FIG. 1, a modular femoral head (B) for surface replacement having a female-type connection portion 1 is illustrated, and the femoral head can be converted into a big femoral head in conventional THA. The female-type connection portion 1 may adopt one of a conically-tapered type 1-C and a rectangularly-tapered type 1-R. A male-type connection portion 11 corresponding to the female-type connection portion 1 and connected with the femoral neck stem may adopt one of a conically-tapered type 11-C and a rectangularly-tapered type 11-R. The male-type connection portion 11 is connected to the female-type connection portion 1 of the femoral head (B). The present invention adopts the conical type or the rectangular type connecting method of the modular FHSR and the big femoral head.

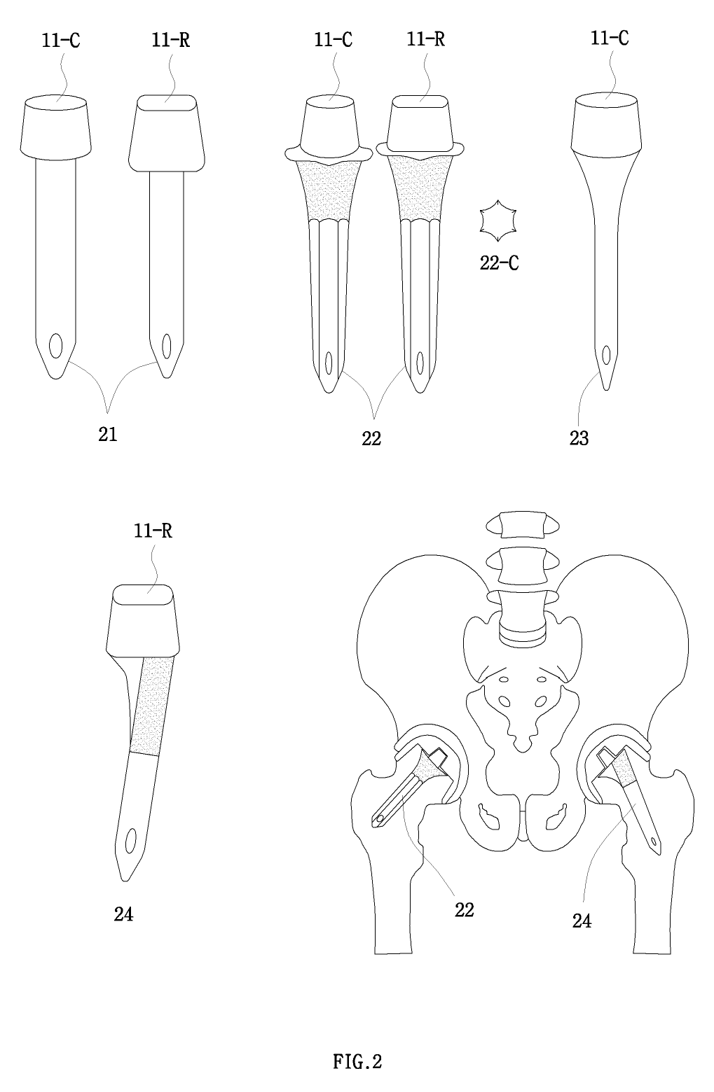

FIG. 2 illustrates a modular femoral neck stem 2, which can be attached to the modular FHSR (B). The modular femoral neck stem 2 may adopt one of various types, such as a straight type 21, a tapered type 22, and a flute type 23, which allows the femoral neck stem to be firmly fixed without rotational deformity at the beginning by attaching a fluted type to the straight type stem. In addition, the modular femoral neck stem 2 may adopt an eccentric type 24, which can fix the femoral head at the center although the femoral neck stem is inserted one-sidedly during operation. So, the modular femoral neck stem 2 according to the present invention can be inserted into the female-type connection portion 1 of the modular femoral head, whereby manufacturing costs can be reduced.

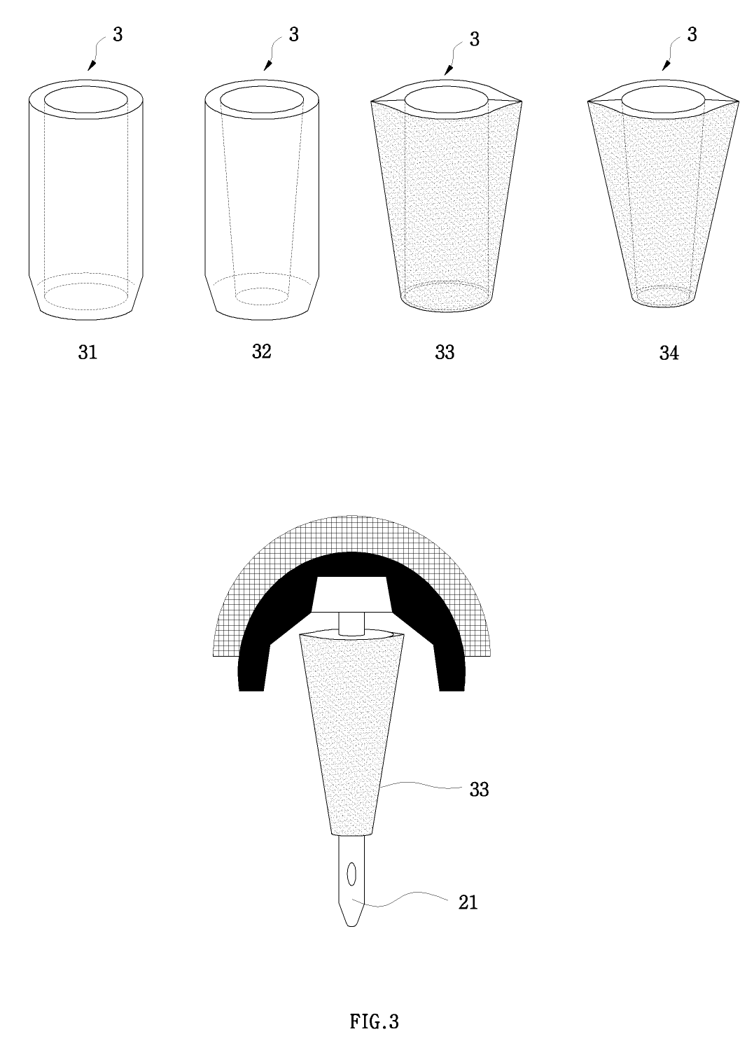

FIG. 3 illustrates sleeves 3 of various types, each of which is fit to the femoral neck stem 2 of the conventional FHSR (A) or modular neck stem also. The sleeve 3 may adopt one of various types, such as a straight type 31, a tapered type 32, a fluted type 33, and a tapered-fluted type 34, so that the sleeve can be used not only in the straight type femoral neck stem 21 but also in the tapered type femoral neck stem 22 as occasion demands.

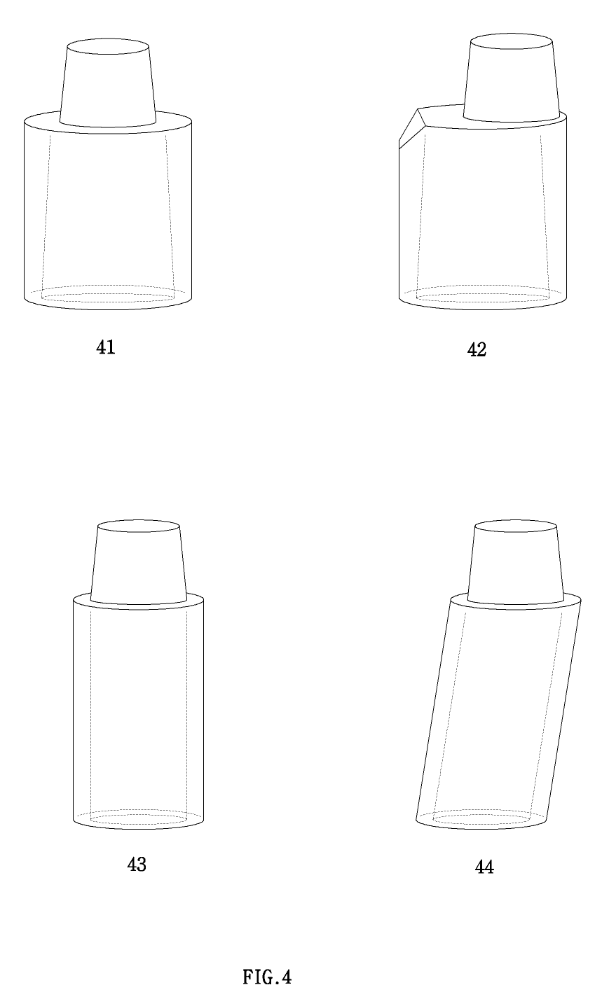

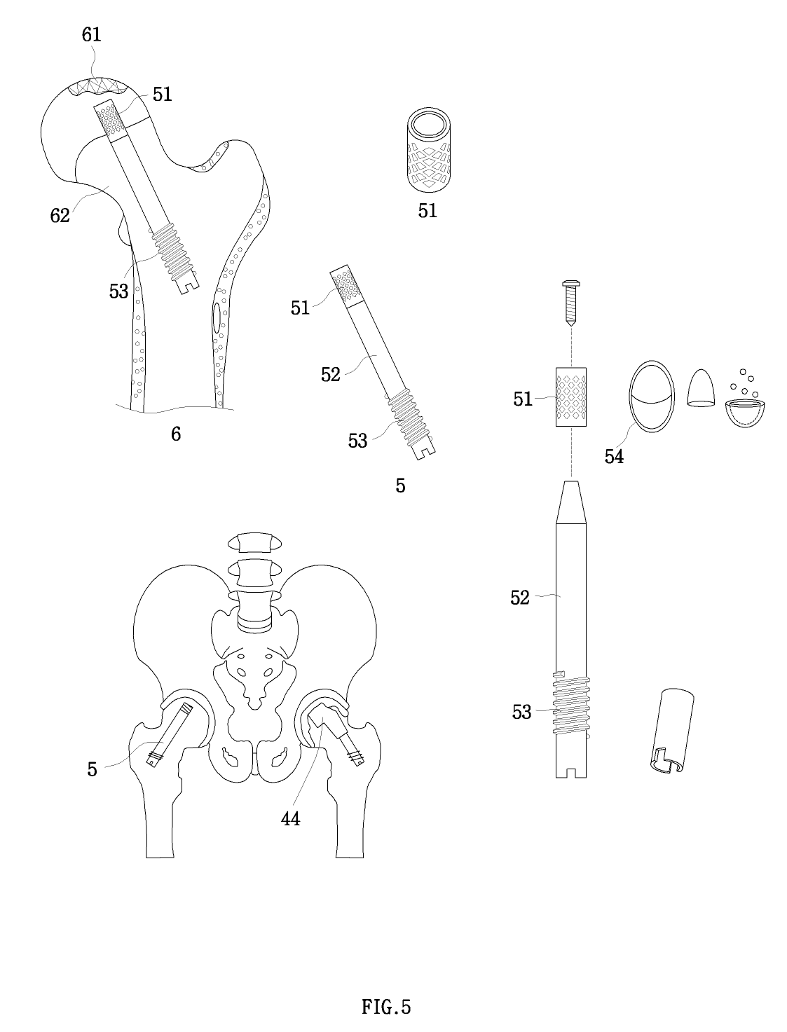

FIG. 4 illustrates an adapter connected to the femoral neck of the conventional THA. The adapter of the conventional THA may also adopt one of a straight type 41 and an eccentric type 42. In addition, FIG. 4 illustrates another adapter connected with an osteoconducting rod 5 (in FIG. 5) used to treat avascular necrosis (hereinafter, called “AVN”), and the adapter connected with the osteoconducting rod 5 may also adopt one of a straight type 43 and an eccentric type 44.

FIG. 5 illustrates the osteoconducting rod 5, which is usable in the beginning stage of AVN. In FIG. 5, the osteoconducting rod 5 is constructed of a cannulated screw type made of Titanium metal. The osteoconducting rod 5 of the metal screw type includes: a shaft 52 of a diameter ranging from 10 mm to 18 mm, the diameter being variable according to sizes of the patients; a screw portion 53 formed at the rear portion thereof; and a Ti-alloy container 51 formed at the front of the shaft. Ti-alloy container for storing BMP family or hydroxyapatite granules for inducing bone on growth during the operation. According to development of the medical science, osteoconducting cells have been developed, but greatest problem in the future is to develop a stem cell carrier or capsule. Since this carrier or capsule which was made of hard gelatin, polylactic acid, or polyglycolic acid. This will be naturally absorbed in the human body, and act as the stem cell carrier or osteoconduction. In FIG. 5, the reference numeral 6 designates a normal femur, 61 designates avascular necrosis of the femoral head, and 62 designates a normal femoral neck.

As described above, the present invention provides the modular FHSR in stead of the conventional fixed type FHSR, and the modular FHSR according to the present invention can be applied in the conventional THA but also to the big femoral head. The various types of FHSR or modular big femoral head which can be selected according to the patients' conditions. As a related part of the FHSR, the sleeves of various types can be applied according to the degree of avascular necrosis of the femoral head to prevent or support a breakage of the femoral neck or femoral neck stem metal as complications of the FHSR The adapter for mounting the big femoral head is connected with the femoral stem of conventional THA in such a way that an artificial femoral head is attached to the femoral neck without removing a healthy femoral neck differently from the conventional total hip replacement even though avascular necrosis of femoral head occurs. Moreover, according to the present invention, by using the adapter connectable with the osteoconducting rod (or a trabecular metal rod), which can be used in the beginning stage of avascular necrosis of the femoral head, even though the avascular necrosis of the femoral head makes progress, total hip replacement of the mini incision method can be performed without inserting the conventional large femoral stem to the marrow cavity, whereby the present invention is economical since it can reduce a surgical operation period and does not need a great deal of blood transfusions, and allow the patients to walk directly after the surgical operation. Owing to the development of the medical science, stem cells can be used experimentally for revascularization of the dead portion of the femoral head, but the greatest problem is to develop a carrier to transfer the stem cells correctly to an avascular lesion without splitting or falling down. To solve such a problem, the present invention 54 act as a stem cell carrier. This container in which we inserted stem cell like a oral pill in medical drug.

While the present invention has been described with reference to the particular illustrative embodiment, it is not to be restricted by the embodiment but only by the appended claims. It is to be appreciated that those skilled in the art can change or modify the embodiment without departing from the scope and spirit of the present invention.

What is claimed is:

1. A modular femoral head surface replacement (FHSR) comprising a femoral head having a female-type connection portion connected with a modular femoral neck stem of total hip arthroplasty (THA), the female-type connection portion being constructed of a conically-tapered type or a rectangular-tapered type.

2. A modular femoral neck stem comprising: a male-type connection portion formed at the front portion thereof and connected with the female-type connection portion of the femoral head; a hole formed at the rear portion thereof for allowing easy removal, insertion and rotation; and a shaft constructed of any one of a straight type, a tapered type, a fluted type and an eccentric type, the male-type connection portion being constructed of a conically-tapered type or a rectangular-tapered type.

3. A sleeve adapted to be fit to a fixed type femoral stem of a conventional FHSR or to a modular femoral stem of the modular FHSR and serving as an auxiliary appliance to provide stability, the sleeve being constructed of any one of a straight type, a tapered type, a fluted type, and a tapered-fluted type.

4. An adapter connectable with the femoral stem of the conventional THA and being constructed of any one of a straight type and an eccentric type, or connectable with an osteoconducting rod used to treat avascular necrosis (AVN) of the femoral head and being also constructed of any one of a straight type and an eccentric type.

5. An osteoconducting rod of a cannulated screw type, which is used to induce osteogenesis or revascularization in a necrosed portion of the femoral head, comprising a container formed at the tip portion thereof for storing stem cells therein.
